(19) United States
(12) Patent Application Publication
(10) Pub. No.: US 2014/0088558 A1
(43) Pub. Date: Mar. 27, 2014

(54) USE OF SWITCHES TO FACILITATE A SAFE AND CONVENIENT ATTACHMENT AND REMOVAL PROCEDURE

(71) Applicant: SANOFI-AVENTIS DEUTSCHLAND GMBH, Frankfurt am Main (DE)

(72) Inventors: Marc Holtwick, Frankfurt am Main (DE); Michael Caspers, Frankfurt am Main (DE); Llona Eggert, Frankfurt am Main (DE)

(21) Appl. No.: 14/119,213

(22) PCT Filed: May 24, 2012
(86) PCT No.: PCT/EP2012/059760

§ 371 Date: Nov. 21, 2013

(30) Foreign Application Priority Data
May 25, 2011 (EP) 11167542.7

(51) Int. Cl. A61M 5/50; A61M 5/19

(52) U.S. Cl. CPC A61M 5/5086; A61M 5/19
USPC 604/506; 604/111

(57) ABSTRACT

The present invention inter alia relates to an apparatus, comprising a detecting arrangement comprising a first and a second detecting unit, wherein the detecting arrangement is configured to at least detect a partial attaching of an attachable unit to the apparatus and a complete attaching of the attachable unit to the apparatus, wherein the attachable unit is configured to be removably attached to the apparatus, and wherein the detecting arrangement is configured to only enable at least one function of the apparatus, when a complete attaching of the attachable unit to the apparatus is detected.

### CROSS REFERENCE TO RELATED APPLICATIONS

The present application is a U.S. National Phase Application pursuant to 35 U.S.C. §371 of International Application No. PCT/EP2012/059760 filed May 24, 2012, which claims priority to European Patent Application No. 11167542.7 filed May 25, 2011. The entire disclosure contents of these applications are herewith incorporated by reference into the present application.

### TECHNICAL FIELD

The present patent application inter alia relates to medical devices of delivering at least two drug agents from separate reservoirs. Such drug agents may comprise a first and a second medicament. The medical device includes a dose setting mechanism for delivering the drug automatically or manually by the user.

### BACKGROUND

The drug agents may be contained in two or more multiple dose reservoirs, containers or packages, each containing independent (single drug compound) or pre-mixed (co-formulated multiple drug compounds) drug agents.

Certain disease states require treatment using one or more different medicaments. Some drug compounds need to be delivered in a specific relationship with each other in order to deliver the optimum therapeutic dose. The present patent application is of particular benefit where combination therapy is desirable, but not possible in a single formulation for reasons such as, but not limited to, stability, compromised therapeutic performance and toxicology.

For example, in some cases it might be beneficial to treat a diabetic with a long acting insulin (also may be referred to as the first or primary medicament) along with a glucagon-like peptide-1 such as GLP-1 or GLP-1 analog (also may be referred to as the second drug or secondary medicament).

Accordingly, there exists a need to provide devices for the delivery of two or more medicaments in a single injection or delivery step that is simple for the user to perform without complicated physical manipulations of the drug delivery device.

### SUMMARY

The proposed drug delivery device provides separate storage containers or cartridge retainers for two or more active drug agents. These active drug agents are then only combined and/or delivered to the patient during a single delivery procedure. These active agents may be administered together in a combined dose or alternatively, these active agents may be combined in a sequential manner, one after the other.

The drug delivery device also allows for the opportunity of varying the quantity of the medicaments. For example, one fluid quantity can be varied by changing the properties of the injection device (e.g., setting a user variable dose or changing the device's “fixed” dose). The second medicament quantity can be changed by manufacturing a variety of secondary drug containing packages with each variant containing a different volume and/or concentration of the second active agent.

The drug delivery device may have a single dispense interface. This interface may be configured for fluid communication with the primary reservoir and with a secondary reservoir of medicament containing at least one drug agent. The drug dispense interface can be a type of outlet that allows the two or more medicaments to exit the system and be delivered to the patient.

The combination of compounds as discrete units or as a mixed unit can be delivered to the body via a double-ended needle assembly. This would provide a combination drug injection system that, from a user's perspective, would be achieved in a manner that closely matches the currently available injection devices that use standard needle assemblies. One possible delivery procedure may involve the following steps:

1. Attach/mount a dispense interface to a distal end of the electro-mechanical injection device. The dispense interface comprises a first and a second proximal needle. The first and second needles pierce a first reservoir containing a primary compound and a second reservoir containing a secondary compound, respectively.

During attaching the dispense interface to the injection device, for instance, the following sequence of steps may at least partially occur:
the attachment means are aligned,
a spring is triggered,
septa of the first and the second reservoir are pierced by the first and second needle, respectively,
a reuse protection such as a lock-out spring of the dispense interface is activated, and
the dispense interface is secured at the injection device via a snap-fit connection.

2. Attach a dose dispenser, such as a double-ended needle assembly, to a distal end of the dispense interface. In this manner, a proximal end of the needle assembly is in fluidic communication with both the primary compound and secondary compound.

3. Dial up/set a desired dose of the primary compound from the injection device, for example, via a graphical user interface (GUI).

4. After the user sets the dose of the primary compound, the micro-processor controlled control unit may determine or compute a dose of the secondary compound and preferably may determine or compute this second dose based on a previously stored therapeutic dose profile. It is this computed combination of medicaments that will then be injected by the user. The therapeutic dose profile may be user selectable.

5. Optionally, after the second dose has been computed, the device may be placed in an armed condition. In such an optional armed condition, this may be achieved by pressing and/or holding an “OK” button on a control panel. This condition may provide for greater than a predefined period of time before the device can be used to dispense the combined dose.

6. Then, the user will insert or apply the distal end of the dose dispenser (e.g., a double ended needle assembly) into the desired injection site. The dose of the combination of the primary compound and the secondary compound (and potentially a third medicament) is administered by activating an injection user interface (e.g., an injection button).

Both medicaments may be delivered via one injection needle or dose dispenser and in one injection step. This offers a convenient benefit to the user in terms of reduced user steps compared to administering two separate injections.

After a specific number of injections (e.g. 1 injection, 3 injections, 5 injections, 10 injections, 20 injections, 50 injections or the like) or after a specific time (e.g. 3 days, 7 days, 14 days or the like) there is a risk for the dose dispenser and/or the dispense interface to be contaminated and, additionally, the tips of the needles of the dose dispenser and/or the dispense interface may be blunted. For instance, a blunted tip of a needle may not be able to sufficiently pierce a septum and/or tissue, for instance inserting a blunted needle in a desired injection site may be very painful. Furthermore, mechanical parts of the dose dispenser and/or the dispense interface such as a valve arrangement may only proper function for a specific number of injections (e.g. 1 injection, 3 injections, 5 injections, 10 injections, 20 injections, 50 injections or the like).

Therefore, the dispense interface may be removable from the injection device. For instance, the dispense interface may be removed from the injection device by pressing a release button.

During removing the dispense interface from the injection device, for instance, a snap-fit connection between the dispense interface and the injection device is released such that the dispense interface moves to a detent position, during this movement the following sequence of steps may at least partially occur:
the needles are removed from the septa so that there is no longer a fluidic connection, and
the reuse protection such as a lock-out spring is activated preventing any reattachment of the dispense interface.

Once in the detent position, the dispense interface can then be manually removed by the user.

However, there are several risks associated with the partial attaching or removing of the dispense interface as well as the potential to circumvent the reuse protection. For instance, a user may reattach the dispense interface by only partially removing it and then trying to push it back on. Re-attachment of the hub carries the risk of drug sitting in the dispense interface indefinitely and potentially being contaminated or degraded.

Therefore, the present invention inter-alia faces the technical problem of mitigating these risks.

According to the present invention, an apparatus comprises a detecting arrangement comprising a first and a second detecting unit, wherein the detecting arrangement is configured to at least detect a partial attaching of an attachable unit to the apparatus and a complete attaching of the attachable unit to the apparatus, wherein the attachable unit is configured to be removably attached to the apparatus, and wherein the detecting arrangement is configured to only enable at least one function of the apparatus, when a complete attaching of the attachable unit to the apparatus is detected.

According to the present invention a method comprises detecting, by a detecting arrangement comprising a first and a second detecting unit, a partial attaching of an attachable unit to an apparatus and a complete attaching of the attachable unit to the apparatus, wherein the attachable unit is configured to be removably attached to the apparatus, and only enabling at least one function of the apparatus, when a complete attaching of the attachable unit to the apparatus is detected.

The apparatus may be a drug delivery device such as a medical device configured to eject a drug agent (e.g. a dose of a medicament) such as an infusion device or an injection device, for instance an insulin injection pen. Injection devices may be used either by medical personnel or by patients themselves. As an example, type-1 and type-2 diabetes may be treated by patients themselves by injection of insulin doses, for example once or several times per day.

For instance, the apparatus is a medical device configured to eject at least two drug agents from separate reservoirs (e.g. cartridges) comprising a first and a second medicament, respectively, but it is not limited thereto. Alternatively, the medical device is for instance a conventional medical device configured to eject a drug agent from a single reservoir (e.g. a single cartridge) such as Applicant's Solostar® insulin injection pen.

The attachable unit may be a (disposable) part attachable to the medical device such as a drug delivery device. For instance, the attachable unit is a dispense interface attachable to a medical device configured to eject a drug agent. A dispense interface may be configured to be in fluid communication with at least one fluid reservoir (e.g. one cartridge) of the medical device containing at least one medicament. For instance, the dispense interface is a type of outlet that allows the at least one medicament to exit the medical device.

The attachable unit is removably attachable to the apparatus. In particular, the dispense interface forming the attachable unit may be attachable and removable from the medical device as described above, but it is not limited thereto.

A partial attaching (or mounting) of an attachable unit to the apparatus for instance corresponds to initiating of attaching the attachable unit to the apparatus. For instance, the apparatus and the attachable unit comprise mating attachment means configured to form a detachable mechanical and/or fluid connection between the apparatus and the attachable unit. Examples of mating attachment means include snap locks, snap fits, snap rings, keyed slots, threads, luer-connectors, canulas, piercable septa and any combinations thereof. For instance, attaching the attachable unit to the apparatus is initiated, when the mating attachment means of the attachable unit and the apparatus are aligned, brought into contact, partially introduced or the like, for instance without forming a (secured) mechanical and/or fluid connection.

A complete attaching of the attachable unit to the apparatus for instance corresponds to the completion of attaching the attachable unit to the apparatus. For instance, attaching the attachable unit to the apparatus is completed, when the mating attachment means of the attachable unit and the apparatus form a (secured) mechanical connection, in particular a secured mechanical and fluid connection. For instance, a connection is secured, when the mating attachment means are in engagement such as snapped in.

The detecting arrangement comprises at least two detecting units, a first and a second detecting unit, wherein the detecting arrangement is configured to at least detect a partial attaching of the attachable unit to the apparatus and a complete attaching of the attachable unit to the apparatus. For instance, the first detecting unit is configured to detect a partial attaching of the attachable unit to the apparatus, and the second detecting unit is configured to detect a complete attaching of the attachable unit to the apparatus. This is inter-alia advantageous to allow to detect whether an attaching is initiated but not completed and, for instance, to advise a user of the apparatus accordingly. In particular, at least the following three situations may be distinguished:
complete removing of the attachable unit from the apparatus (e.g. no connection at all),
initiated/partial attaching of the attachable unit to the apparatus (e.g. no secured connection), and
completed attaching of the attachable unit to the apparatus (e.g. secured connection).

The detecting arrangement is configured to only enable at least one function of the apparatus, when a complete attaching of the attachable unit to the apparatus is detected. The detecting arrangement may be a mechanical arrangement such as a micro-mechanical arrangement, an electronic arrangement and/or a combination thereof. For instance, the detecting arrangement is formed from a (micro-) mechanical arrangement mechanically disabling (e.g. blocking) the at least one function of the apparatus, when a complete attaching of the attachable unit to the apparatus is not detected. Alternatively or additionally, the detecting arrangement may at least partially be formed from an (micro-) electronic arrangement. For instance, the detecting arrangement may at least partially be implemented in a processing unit of the apparatus such that the at least one function of the apparatus is electrically and/or logically disabled, when a complete attaching of the attachable unit to the apparatus is not detected. This is inter-alia advantageous to allow to only enable the at least one function of the apparatus, when there is a secure connection between the apparatus and the attachable unit. For instance, the correct function of the apparatus and/or of the at least one function may depend on such a secure connection.

The processing unit such as a micro-processor control unit is for instance a microprocessor, a Digital Signal Processor (DSP), an Application Specific Integrated Circuit (ASIC), a Field Programmable Gate Array (FPGA) or the like. The processing unit may execute program code (e.g. software or firmware) stored in a program memory, and uses a main memory, for instance to store intermediate results. For instance, the program memory may comprise a computer program having program code for performing the method according to the present invention when the computer program is executed on the processing unit. The computer program may for instance be distributable via a network, such as for instance the Internet. The computer program may for instance be storable or encodable in a computer-readable medium.

The processing unit may be configured to communication with the first and the second detecting units, for instance the processing unit may be configured to receive signals from the detecting unit, the signals representing a complete removing, a partial attaching and a complete attaching.

In the example in which the apparatus is a medical device configured to eject a medicament and the attachable unit is a dispense interface attachable thereto, a correct ejection of a selected dose of the medicament via the dispense interface may only be guaranteed, when a secure mechanical and fluid connection is formed between the dispense interface and the medical device. In this example, the dose selection and/or the ejection of the medicament may only be enabled by the detecting arrangement, when a complete attaching of the attachable unit to the apparatus forming a secure mechanical and fluid connection is detected. Furthermore, a partial removing of an attached dispense interface and a reattaching of the only partially removed dispense interface may also be detected and, in this situation, the dose selection and/or the ejection of the medicament may be not enabled by the detecting arrangement.

As described above, there are several risks associated with the partial attaching or removing of the attachable unit such as a dispense interface. To effectively mitigate these risks, the apparatus comprises the detecting arrangement comprising at least two detecting units.

In the following, features and embodiments (exhibiting further features) of the present invention will be described, which are understood to equally apply to the apparatus and the method as described above. These single features/embodiments are considered to be exemplary and non-limiting, and to be respectively combinable independently from other disclosed features/embodiments of the apparatus and the method as described above. Nevertheless, these features/embodiments shall also be considered to be disclosed in all possible combinations with each other and with the apparatus and the method as described above. For instance, a mentioning that an apparatus according to the present invention is configured to perform a certain action should be understood to also disclose an according method step of the method according to the present invention.

According to an embodiment of the present invention, the first detecting unit is activated, when the attachable unit is at least partially attached to the apparatus, and the second detecting unit is activated, when the attachable unit is completely attached to the apparatus.

For instance, the first detecting unit is arranged in the apparatus such that it is activated early during attaching of the attachable unit to the apparatus signalling that attaching is initiated. For instance, the first detecting unit remains activated until the attachable unit is completely removed from the apparatus. For instance, the second detecting unit is arranged in the apparatus such that, only when the attachment means form a (secure) connection such as a mechanical and/or fluid connection between the apparatus and the attachable unit, the second detecting unit is activated signalling that attaching is completed.

This is inter-alia advantageous to allow to detect whether an attaching is initiated but not completed and, for instance, to advise a user of the apparatus accordingly. In particular, at least the following three situations may be distinguished:
complete removing of the attachable unit from the apparatus (e.g. no connection at all),
initiated/partial attaching of the attachable unit to the apparatus (e.g. no secured connection), and
complete attaching of the attachable unit to the apparatus (e.g. secured connection).

According to an embodiment of the present invention, the detecting arrangement is configured to only enable the at least one function of the apparatus, when the first detecting unit and the second detecting unit are activated, for instance subsequently and/or simultaneously activated.

For instance the first and second detecting unit are subsequently activated, when attaching the attachable unit to the apparatus, and/or the first and second detecting unit are simultaneously activated, when the attachable unit is completely attached to the apparatus.

This is inter-alia advantageous to allow to only enable the at least one function of the apparatus, when there is a secure connection between the apparatus and the attachable unit.

According to an embodiment of the present invention, the detecting arrangement is configured to detect a partial removing of the attachable unit from the apparatus and a complete removing of the attachable unit from the apparatus, and the detecting arrangement is configured to disable the at least one function of the apparatus, when at least the partial removing of the attachable unit from the apparatus is detected.

For instance, the second detecting unit is configured to detect a partial removing of the attachable unit from the apparatus, and the first detecting unit is configured to detect a complete removing of the attachable unit to the apparatus. This is inter-alia advantageous to allow to detect whether a removing is initiated but not completed and, for instance, to advise a user of the apparatus accordingly. In particular, at least the following three situations may be distinguished:
complete attaching of the attachable unit to the apparatus (e.g. secured connection)
initiated/partial removing of the attachable unit from the apparatus (e.g. no secured connection), and
complete removing of the attachable unit from the apparatus (e.g. no connection at all).

According to an embodiment of the present invention, the second detecting unit is deactivated, when the attachable unit is at least partially removed from the apparatus, and the first detecting unit is deactivated, when the attachable unit is completely removed from the apparatus.

For instance, the second detecting unit is arranged in the apparatus such that it is deactivated early during removing of the attachable unit from the apparatus signalling that removing is initiated. For instance, the first detecting unit is arranged in the apparatus such that, only when the removing is completed, the first detecting unit is deactivated signalling that removing is completed.

As described above, this is inter-alia advantageous to allow to detect whether a removing is initiated but not completed and, for instance, to advise a user of the apparatus accordingly.

According to an embodiment of the present invention, the detecting arrangement is configured to disable the at least one function of the apparatus, when the second detecting unit is deactivated. This is inter-alia advantageous to allow to only enable the at least one function of the apparatus, when there is a secure connection between the apparatus and the attachable unit, and to disable the at least one function otherwise.

According to an embodiment of the present invention, the detecting arrangement is configured to only enable the at least one function of the apparatus after deactivating the second detecting unit, when subsequently the first detecting unit is deactivated, and when subsequently the first and the second detecting unit are activated. For instance, the detecting arrangement is configured to only enable the at least one function of the apparatus after complete removing of an attachable unit from the apparatus and a subsequent complete attaching of an attachable unit to the apparatus.

This embodiment is inter-alia advantageous to prevent circumventing a reuse protection mechanism such as a lock-out spring of the attachable unit preventing a reattaching of the attachable unit to the apparatus.

For instance, the attachable unit is a disposable part such as a dispense interface that is to be only used for a pre-defined period of time and/or usages. For instance, after a specific number of ejections (e.g. 1 ejection, 3 ejections, 5 ejections, 10 ejections, 20 ejections, 50 ejections or the like) or after a specific time (e.g. 1 day, 3 days, 7 days, 14 days, or the like) there is a risk for a dispense interface attached to a medical device configured to eject a medicament to be contaminated. Furthermore, mechanical parts of the dispense interface such as a valve arrangement may only proper function for a specific number of ejections (e.g. 1 ejection, 3 ejections, 5 ejections, 10 ejections, 20 ejections, 50 ejections or the like). Therefore, the dispense interface may necessarily be removed from the medical device configured to eject a medicament after a pre-defined period of time and/or number of ejections.

For instance, the medical device may require the user to remove the dispense interface after the pre-defined period of time and/or number of ejections, and, to prevent reattaching of the dispense interface to the medical device, the dispense interface may comprise a reuse protection mechanism, for instance mechanically preventing a reattaching. The reuse protection mechanism is for instance activated, when the dispense interface is (completely) removed from the medical device. In this example, only enabling the dose selection and/or the ejection of the medicament, when the (old) dispense interface is completely removed from the medical device and, subsequently, a (new) dispense interface is completely attached to the medical device prevents reattaching of the (old) dispense interface.

According to an embodiment of the present invention, the first and/or the second detecting unit is at least one of an optical sensor, contact sensor and a proximity sensor. An optical sensor is for instance a charge coupled device, a photo diode, a photo resistor, a photo transistor, an infrared sensor or the like. A contact sensor is for instance a contact switch, a pressure activated switch or the like. A proximity sensor is for instance a reed switch, a touch switch such as a capacitance or resistance touch switch or the like.

For instance, the type and position of the first detecting unit is chosen such that it can sense initiating attaching of the attachable unit to the apparatus, and the type and position of the second detecting unit may be chosen such that it can sense complete attaching of the attachable unit to the apparatus. For instance, the type and position of the first detecting unit is chosen such that it can sense complete removing of the attachable unit from the apparatus, and the type and position of the second detecting unit may be chosen such that it can sense initiating removing of the attachable unit from the apparatus.

For instance, attaching the attachable unit to the apparatus is initiated, when the mating attachment means of the attachable unit and the apparatus are aligned, brought into contact, partially introduced or the like. For instance, the first detecting unit is a proximity sensor arranged at a distal position at the surface of the attachment means of the apparatus such that it is activated signalling initiated attaching, when the mating attachment means of the attachable unit and the apparatus are brought into contact, and deactivated signalling a complete removing, when the mating attachment means are completely removed from each other.

For instance, attaching the attachable unit to the apparatus is completed, when the mating attachment means of the attachable unit and the apparatus form a (secured) connection, in particular a secured mechanical and fluid connection. For instance, the second detecting unit is a proximity sensor arranged at a proximal position at the surface of the attachment means of the apparatus such that it is activated signalling complete attaching, when the mating attachment means of the attachable unit and the apparatus are in engagement, and deactivated signalling a partial removing, when the mating attachment means are removed from engagement.

The distal position may be closer at a distal end of the attachment means than the proximal position.

For instance, when the attachable unit is attached to the apparatus, a surface of the attachable unit may slide along a surface of the apparatus. When the attaching is initiated the surface of the attachable unit may only cover a distal portion of a surface of the apparatus. When the attaching is completed the surface of the attachable unit may also cover a proximal portion of the surface of the apparatus. For instance, the first detecting unit may be arranged at the distal portion of the surface of the apparatus and the second detecting unit may be positioned at the proximal portion of the surface of the apparatus.

According to an embodiment of the present invention, the detecting arrangement is configured to activate a warning, when a partial attaching and/or a partial removing of the attachable unit is detected. For instance, the detecting arrangement may be configured to display a warning message on a displaying unit of the apparatus and/or to generate an acoustical warning message. For instance, the user is advised to completely attach and/or remove the attachable unit. This embodiment is particularly advantageous to support the user, when attaching and/or removing the attachable unit.

According to an embodiment of the present invention, the detecting arrangement comprises a movable element configured to be moved from a first position at the apparatus to a second position at the apparatus during attaching of the attachable unit to the apparatus and/or to be moved from the second position to the first position during removing of the attachable unit from the apparatus. The movable element is for instance relatively to the apparatus and/or at least in one direction (e.g. longitudinally) movable. For instance, the movable element is a part of the apparatus. Alternatively, the movable element is a part of the attachable unit. The movable element may be a push rod.

For instance, the attachable unit causes (e.g. pushes) the movable element to be moved from the first to the second position, when the attachable unit is attached to the apparatus. For instance, the attachable unit causes (e.g. pulls) the movable element to be moved from the second to the first position, when the attachable unit is removed from the apparatus. For instance, the movable element is at least partially arranged in the apparatus.

When the movable element is in the first position, the attachable unit may be completely removed from the apparatus; when the movable element is between the first and the second position, the attachable unit may be partially attached to the apparatus and/or partially removed from the apparatus; and when the movable element is in the second position, the attachable unit may be completely attached to the apparatus.

This embodiment is inter-alia advantageous to allow to detect whether an attaching and/or removing is initiated but not completed depending on the position of the movable element. In particular, at least the following three situations may be distinguished:
complete attaching of the attachable unit to the apparatus (e.g. secured connection)
initiated/partial removing of the attachable unit from the apparatus and/or initiated/partial attaching of the attachable unit to the apparatus (e.g. no secured connection), and
complete removing of the attachable unit to the apparatus (e.g. no connection at all).

According to an embodiment of the present invention, the movable element is spring loaded at least in the second position. For instance, the movable element is resiliently held and movably arranged in the apparatus. This embodiment is inter-alia advantageous to cause the movable element to move from the second to the first position, when the attachable unit is removed from the apparatus.

According to an embodiment of the present invention, the first and the second detecting unit are deactivated, when the movable element is in the first position, the first detecting unit is activated and the second detecting unit is deactivated, when the movable element is positioned between the first and the second position, and the first and second detecting unit are activated, when the movable element is in the second position.

For instance, the first detecting unit is positioned between the first and the second position, and the second detecting unit is positioned at the second position. In particular, the first detecting unit may be positioned in the apparatus at a distal position which is not covered by (an activating portion of) the movable element, when it is positioned at the first position, but which is covered by (an activating portion of) the movable element, when it is positioned between the first and the second position. In particular, the second detecting unit may be positioned in the apparatus at a proximal position which is only covered by the movable element positioned at the second position.

This embodiment is inter-alia advantageous to allow to position the detecting units at positions within the apparatus and/or spaced from the attachment means, for instance secure positions and/or positions having more installation space.

According to an embodiment of the present invention, the apparatus is a medical device configured to eject a medicament and/or the attachable unit is a dispense interface or a needle assembly attachable to the medical device.

According to an embodiment of the present invention, the at least one function of the apparatus is selecting a dose of the medicament and/or ejecting the medicament. This embodiment is inter-alia advantageous to prevent ejecting a medicament, when the dispense interface is not securely connected to the medical device.

These as well as other advantages of various aspects of the present invention will become apparent to those of ordinary skill in the art by reading the following detailed description, with appropriate reference to the accompanying drawings.

### BRIEF DESCRIPTION OF THE FIGURES

FIG. 1 illustrates a perspective view of the delivery device illustrated in FIG. 1a and 1b with an end cap of the device removed;

FIG. 2 illustrates a perspective view of the delivery device distal end showing the cartridge;

FIG. 3 illustrates a perspective view of the cartridge holder illustrated in FIG. 1 with one cartridge retainer in an open position;

FIG. 4 illustrates a dispense interface and a dose dispenser that may be removably mounted on a distal end of the delivery device illustrated in FIG. 1;

FIG. 5 illustrates the dispense interface and the dose dispenser illustrated in FIG. 4 mounted on a distal end of the delivery device illustrated in FIG. 1;

FIG. 6 illustrates one arrangement of the dose dispenser that may be mounted on a distal end of the delivery device;

FIG. 7 illustrates a perspective view of the dispense interface illustrated in FIG. 4;

FIG. 8 illustrates another perspective view of the dispense interface illustrated in FIG. 4;

FIG. 9 illustrates a cross-sectional view of the dispense interface illustrated in FIG. 4;

FIG. 10 illustrates an exploded view of the dispense interface illustrated in FIG. 4;

FIG. 11 illustrates a cross-sectional view of the dispense interface and dose dispenser mounted onto a drug delivery device, such as the device illustrated in FIG. 1;

FIG. 12 illustrates a cross-sectional view of the attaching of the dispense interface onto a drug delivery device, such as the device illustrated in FIG. 1;

FIG. 13 illustrates a method for attaching the dispense interface to a drug delivery device, such as the device illustrated in FIG. 1; and

FIG. 14 illustrates a method for removing the dispense interface from a drug delivery device, such as the device illustrated in FIG. 1.

### DETAILED DESCRIPTION

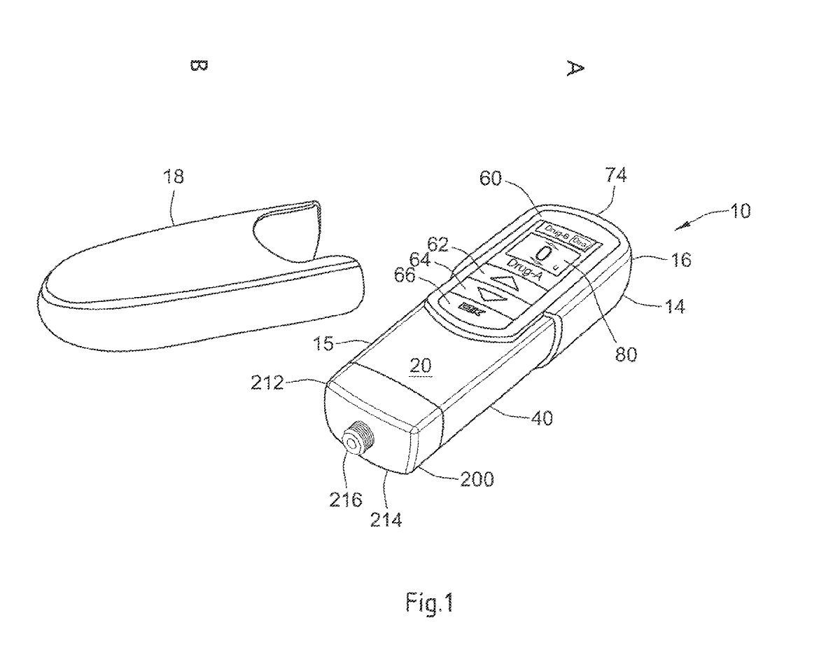

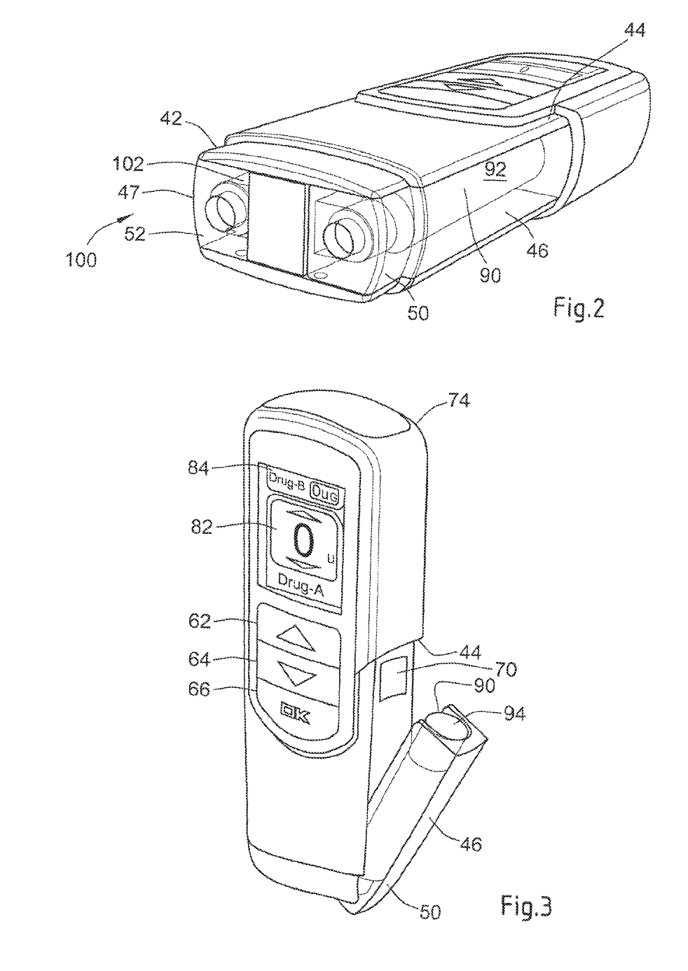

The drug delivery device illustrated in FIG. 1 comprises a main body 14 that extends from a proximal end 16 to a distal end 15. At the distal end 15, a removable end cap or cover 18 is provided. This end cap 18 and the distal end 15 of the main body 14 work together to provide a snap fit or form fit connection so that once the cover 18 is slid onto the distal end 15 of the main body 14, this frictional fit between the cap and the main body outer surface 20 prevents the cover from inadvertently falling off the main body.

The main body 14 contains a micro-processor control unit, an electro-mechanical drive train, and at least two medicament reservoirs. When the end cap or cover 18 is removed from the device 10 (as illustrated in FIG. 1), a dispense interface 200 is mounted to the distal end 15 of the main body 14, and a dose dispenser (e.g., a needle assembly) is attached to the interface. The drug delivery device 10 can be used to administer a computed dose of a second medicament (secondary drug compound) and a variable dose of a first medicament (primary drug compound) through a single needle assembly, such as a double ended needle assembly.

A control panel region 60 is provided near the proximal end of the main body 14. Preferably, this control panel region 60 comprises a digital display 80 along with a plurality of human interface elements that can be manipulated by a user to set and inject a combined dose. In this arrangement, the control panel region comprises a first dose setting button 62, a second dose setting button 64 and a third button 66 designated with the symbol “OK.” In addition, along the most proximal end of the main body, an injection button 74 is also provided (not visible in the perspective view of FIG. 1).

The cartridge holder 40 can be removably attached to the main body 14 and may contain at least two cartridge retainers 50 and 52. Each retainer is configured so as to contain one medicament reservoir, such as a glass cartridge. Preferably, each cartridge contains a different medicament.

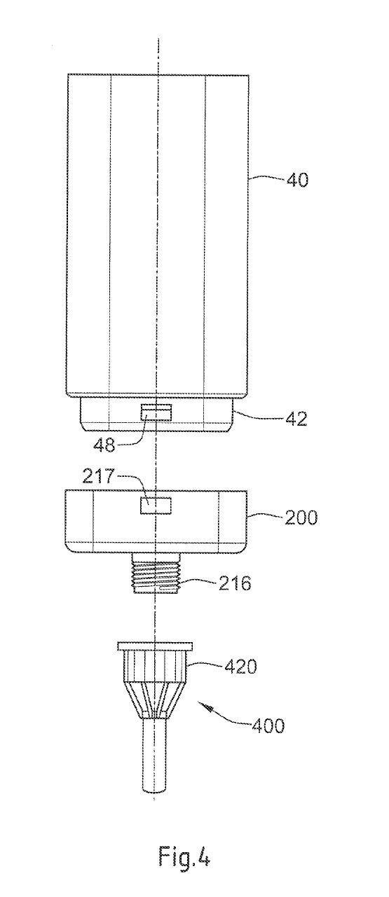

In addition, at the distal end of the cartridge holder 40, the drug delivery device illustrated in FIG. 1 includes a dispense interface 200. As will be described in relation to FIG. 4, in one arrangement, this dispense interface 200 includes a main outer body 212 that is removably attached to a distal end 42 of the cartridge housing 40. As can be seen in FIG. 1, a distal end 214 of the dispense interface 200 preferably comprises a needle hub 216. This needle hub 216 may be configured so as to allow a dose dispenser, such as a conventional pen type injection needle assembly, to be removably mounted to the drug delivery device 10.

Once the device is turned on, the digital display 80 shown in FIG. 1 illuminates and provides the user certain device information, preferably information relating to the medicaments contained within the cartridge holder 40. For example, the user is provided with certain information relating to both the primary medicament (Drug A) and the secondary medicament (Drug B).

As shown in FIG. 3, the first and a second cartridge retainers 50, 52 comprise hinged cartridge retainers. These hinged retainers allow user access to the cartridges. FIG. 3 illustrates a perspective view of the cartridge holder 40 illustrated in FIG. 1 with the first hinged cartridge retainer 50 in an open position. FIG. 3 illustrates how a user might access the first cartridge 90 by opening up the first retainer 50 and thereby having access to the first cartridge 90.

As mentioned above when discussing FIG. 1, a dispense interface 200 is coupled to the distal end of the cartridge holder 40. FIG. 4 illustrates a flat view of the dispense interface 200 unconnected to the distal end of the cartridge holder 40. A dose dispenser or needle assembly that may be used with the interface 200 is also illustrated and is provided in a protective outer cap 420.

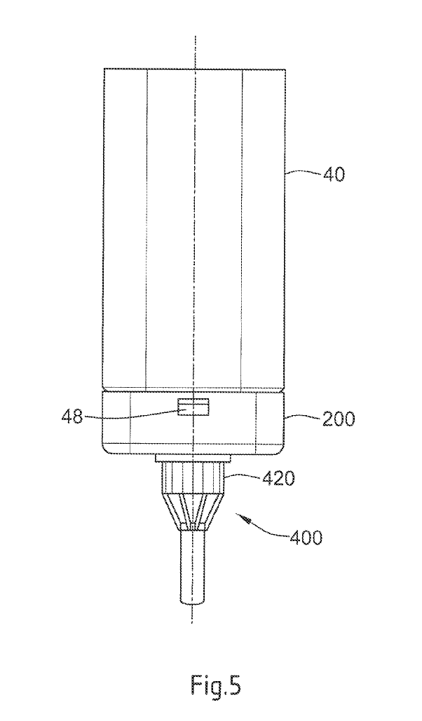

In FIG. 5, the dispense interface 200 illustrated in FIG. 4 is shown coupled to the cartridge holder 40. The axial attachment means between the dispense interface 200 and the cartridge holder 40 can be any known axial attachment means to those skilled in the art, including snap locks, snap fits, snap rings, keyed slots, and combinations of such connections. The connection or attachment between the dispense interface and the cartridge holder may also contain additional features (not shown), such as connectors, stops, splines, ribs, grooves, pips, clips and the like design features, that ensure that specific hubs are attachable only to matching drug delivery devices. Such additional features would prevent the insertion of a non-appropriate secondary cartridge to a non-matching injection device.

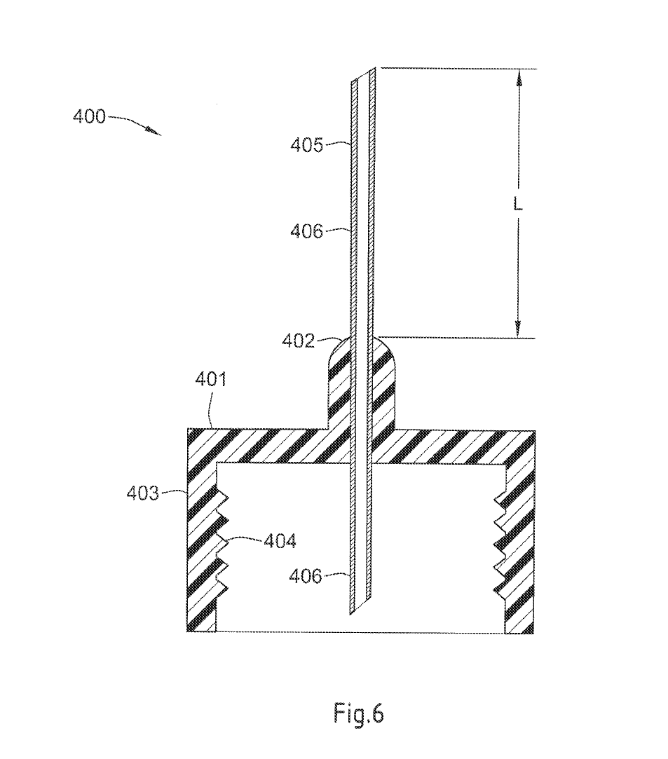

FIG. 5 also illustrates the needle assembly 400 and protective cover 420 coupled to the distal end of the dispense interface 200 that may be screwed onto the needle hub of the interface 200. FIG. 6 illustrates a cross sectional view of the double ended needle assembly 402 mounted on the dispense interface 200 in FIG. 5.

The needle assembly 400 illustrated in FIG. 6 comprises a double ended needle 406 and a hub 401. The double ended needle or cannula 406 is fixedly mounted in a needle hub 401. This needle hub 401 comprises a circular disk shaped element which has along its periphery a circumferential depending sleeve 403. Along an inner wall of this hub member 401, a thread 404 is provided. This thread 404 allows the needle hub 401 to be screwed onto the dispense interface 200 which, in one preferred arrangement, is provided with a corresponding outer thread along a distal hub. At a center portion of the hub element 401 there is provided a protrusion 402. This protrusion 402 projects from the hub in an opposite direction of the sleeve member. A double ended needle 406 is mounted centrally through the protrusion 402 and the needle hub 401. This double ended needle 406 is mounted such that a first or distal piercing end 405 of the double ended needle forms an injecting part for piercing an injection site (e.g., the skin of a user).

Similarly, a second or proximal piercing end 406 of the needle assembly 400 protrudes from an opposite side of the circular disc so that it is concentrically surrounded by the sleeve 403. In one needle assembly arrangement, the second or proximal piercing end 406 may be shorter than the sleeve 403 so that this sleeve to some extent protects the pointed end of the back sleeve. The needle cover cap 420 illustrated in FIGS. 4 and 5 provides a form fit around the outer surface 403 of the hub 401.

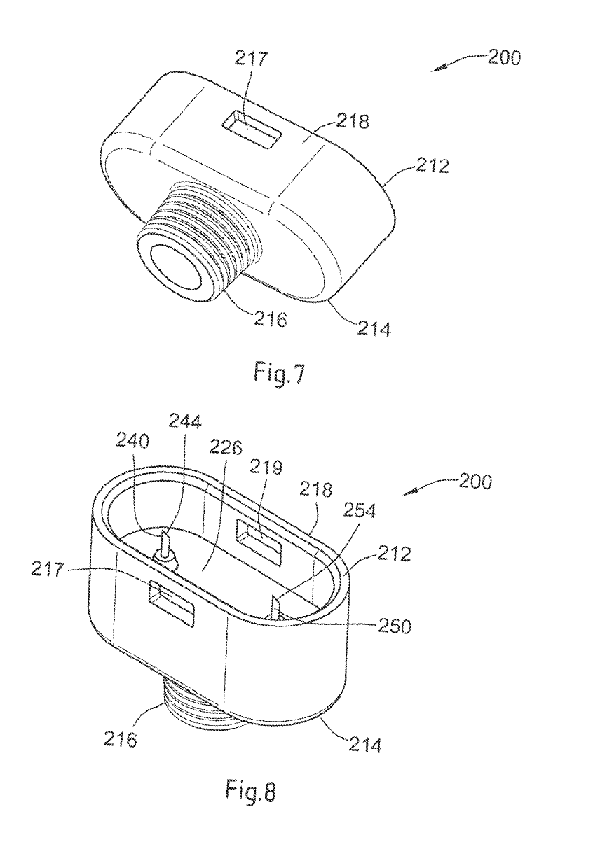

Referring now to FIGS. 4 to 11, one preferred arrangement of this interface 200 will now be discussed. In this one preferred arrangement, this interface 200 comprises:
a. a main outer body 210,
b. an first inner body 220,
c. a second inner body 230,
d. a first piercing needle 240,
e. a second piercing needle 250,
f. a valve seal 260, and
g. a septum 270.

The main outer body 210 comprises a main body proximal end 212 and a main body distal end 214. At the proximal end 212 of the outer body 210, a connecting member is configured so as to allow the dispense interface 200 to be attached to the distal end of the cartridge holder 40. Preferably, the connecting member is configured so as to allow the dispense interface 200 to be removably connected the cartridge holder 40. In one preferred interface arrangement, the proximal end of the interface 200 is configured with an upwardly extending wall 218 having at least one recess. For example, as may be seen from FIG. 8, the upwardly extending wall 218 comprises at least a first recess 217 and a second recess 219.

Preferably, the first and the second recesses 217, 219 are positioned within this main outer body wall so as to cooperate with an outwardly protruding member located near the distal end of the cartridge housing 40 of the drug delivery device 10. For example, this outwardly protruding member 48 of the cartridge housing may be seen in FIGS. 4 and 5. A second similar protruding member is provided on the opposite side of the cartridge housing. As such, when the interface 200 is axially slid over the distal end of the cartridge housing 40, the outwardly protruding members will cooperate with the first and second recess 217, 219 to form an interference fit, form fit, or snap lock. Alternatively, and as those of skill in the art will recognize, any other similar connection mechanism that allows for the dispense interface and the cartridge housing 40 to be axially coupled could be used as well.

The main outer body 210 and the distal end of the cartridge holder 40 act to form an axially engaging snap lock or snap fit arrangement that could be axially slid onto the distal end of the cartridge housing. In one alternative arrangement, the dispense interface 200 may be provided with a coding feature so as to prevent inadvertent dispense interface cross use. That is, the inner body of the hub could be geometrically configured so as to prevent an inadvertent cross use of one or more dispense interfaces.

A mounting hub is provided at a distal end of the main outer body 210 of the dispense interface 200. Such a mounting hub can be configured to be releasably connected to a needle assembly. As just one example, this connecting means 216 may comprise an outer thread that engages an inner thread provided along an inner wall surface of a needle hub of a needle assembly, such as the needle assembly 400 illustrated in FIG. 6. Alternative releasable connectors may also be provided such as a snap lock, a snap lock released through threads, a bayonet lock, a form fit, or other similar connection arrangements.

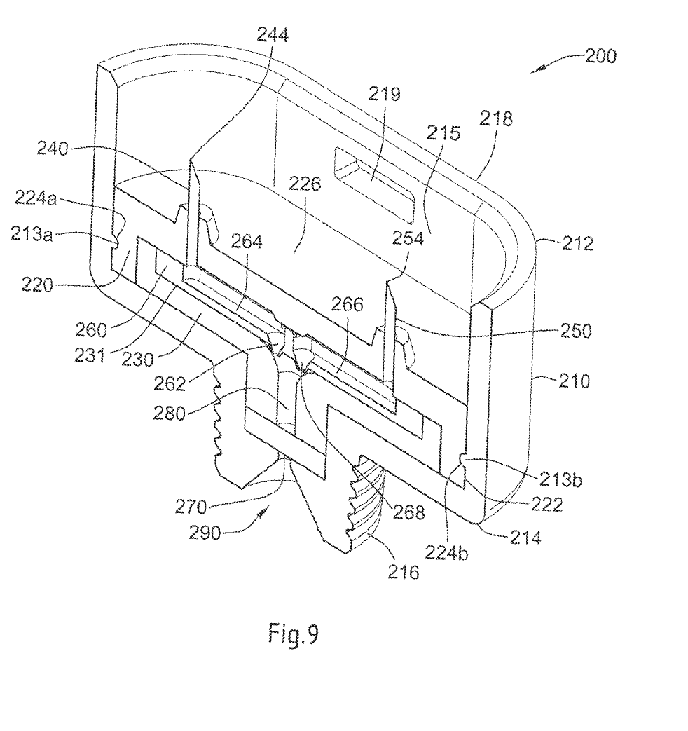

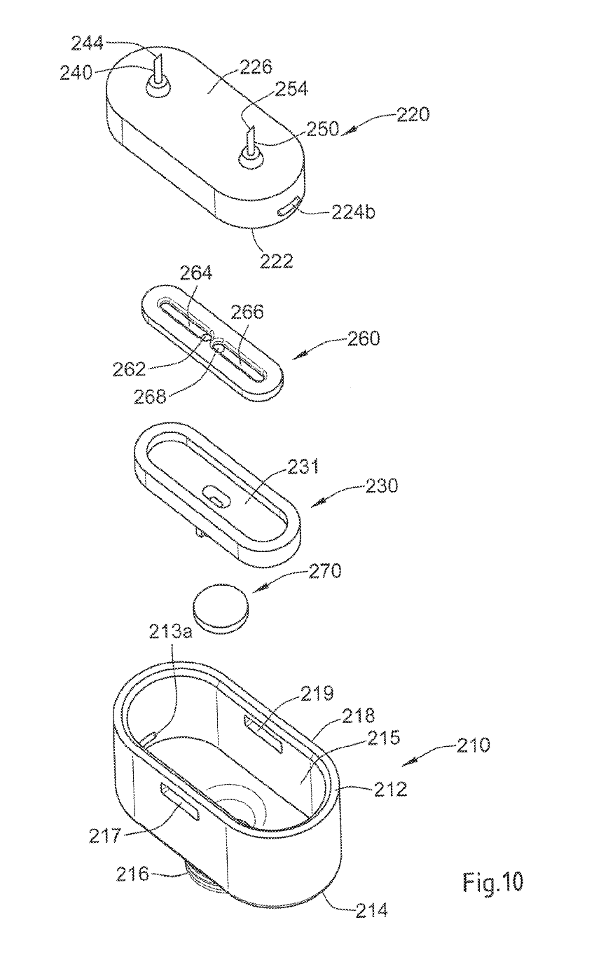

The dispense interface 200 further comprises a first inner body 220. Certain details of this inner body are illustrated in FIG. 8-11. Preferably, this first inner body 220 is coupled to an inner surface 215 of the extending wall 218 of the main outer body 210. More preferably, this first inner body 220 is coupled by way of a rib and groove form fit arrangement to an inner surface of the outer body 210. For example, as can be seen from FIG. 9, the extending wall 218 of the main outer body 210 is provided with a first rib 213a and a second rib 213b. This first rib 213a is also illustrated in FIG. 10. These ribs 213a and 213b are positioned along the inner surface 215 of the wall 218 of the outer body 210 and create a form fit or snap lock engagement with cooperating grooves 224a and 224b of the first inner body 220. In a preferred arrangement, these cooperating grooves 224a and 224b are provided along an outer surface 222 of the first inner body 220.

In addition, as can be seen in FIG. 8-10, a proximal surface 226 near the proximal end of the first inner body 220 may be configured with at least a first proximally positioned piercing needle 240 comprising a proximal piercing end portion 244. Similarly, the first inner body 220 is configured with a second proximally positioned piercing needle 250 comprising a proximally piercing end portion 254. Both the first and second needles 240, 250 are rigidly mounted on the proximal surface 226 of the first inner body 220.

Preferably, this dispense interface 200 further comprises a valve arrangement. Such a valve arrangement could be constructed so as to prevent cross contamination of the first and second medicaments contained in the first and second reservoirs, respectively. A preferred valve arrangement may also be configured so as to prevent back flow and cross contamination of the first and second medicaments.

In one preferred system, dispense interface 200 includes a valve arrangement in the form of a valve seal 260. Such a valve seal 260 may be provided within a cavity 231 defined by the second inner body 230, so as to form a holding chamber 280. Preferably, cavity 231 resides along an upper surface of the second inner body 230. This valve seal comprises an upper surface that defines both a first fluid groove 264 and second fluid groove 266. For example, FIG. 9 illustrates the position of the valve seal 260, seated between the first inner body 220 and the second inner body 230. During an injection step, this seal valve 260 helps to prevent the primary medicament in the first pathway from migrating to the secondary medicament in the second pathway, while also preventing the secondary medicament in the second pathway from migrating to the primary medicament in the first pathway. Preferably, this seal valve 260 comprises a first non-return valve 262 and a second non-return valve 268. As such, the first non-return valve 262 prevents fluid transferring along the first fluid pathway 264, for example a groove in the seal valve 260, from returning back into this pathway 264. Similarly, the second non-return valve 268 prevents fluid transferring along the second fluid pathway 266 from returning back into this pathway 266.

Together, the first and second grooves 264, 266 converge towards the non-return valves 262 and 268 respectively, to then provide for an output fluid path or a holding chamber 280. This holding chamber 280 is defined by an inner chamber defined by a distal end of the second inner body both the first and the second non return valves 262, 268 along with a pierceable septum 270. As illustrated, this pierceable septum 270 is positioned between a distal end portion of the second inner body 230 and an inner surface defined by the needle hub of the main outer body 210.

The holding chamber 280 terminates at an outlet port of the interface 200. This outlet port 290 is preferably centrally located in the needle hub of the interface 200 and assists in maintaining the pierceable seal 270 in a stationary position. As such, when a double ended needle assembly is attached to the needle hub of the interface (such as the double ended needle illustrated in FIG. 6), the output fluid path allows both medicaments to be in fluid communication with the attached needle assembly.

The hub interface 200 further comprises a second inner body 230. As can be seen from FIG. 9, this second inner body 230 has an upper surface that defines a recess, and the valve seal 260 is positioned within this recess. Therefore, when the interface 200 is assembled as shown in FIG. 9, the second inner body 230 will be positioned between a distal end of the outer body 210 and the first inner body 220. Together, second inner body 230 and the main outer body hold the septum 270 in place. The distal end of the inner body 230 may also form a cavity or holding chamber that can be configured to be fluid communication with both the first groove 264 and the second groove 266 of the valve seal.

Axially sliding the main outer body 210 over the distal end of the drug delivery device attaches the dispense interface 200 to the multi-use device. In this manner, a fluid communication may be created between the first needle 240 and the second needle 250 with the primary medicament of the first cartridge and the secondary medicament of the second cartridge, respectively.

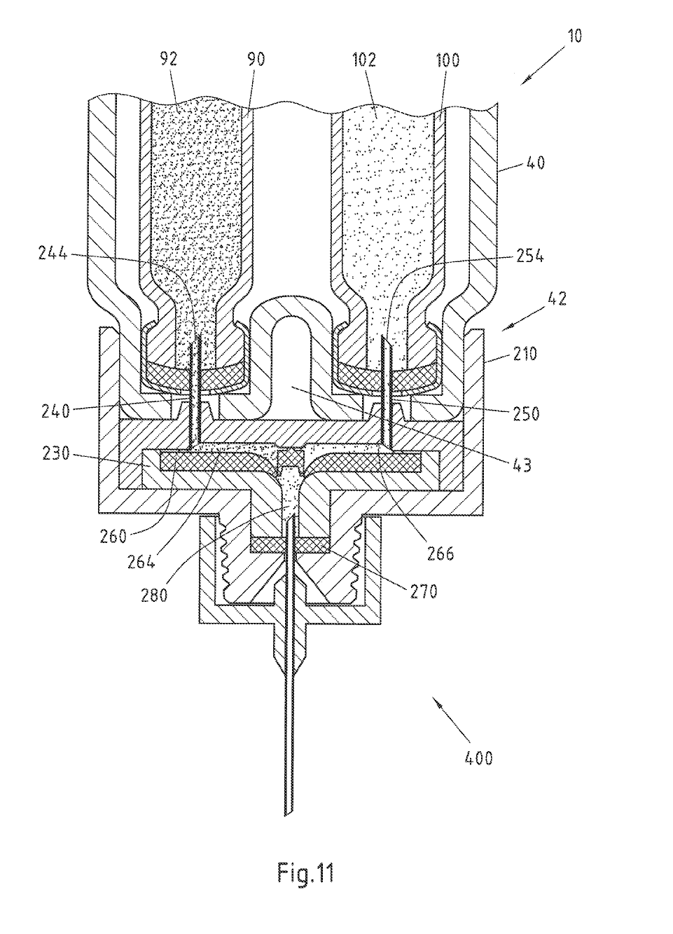

FIG. 11 illustrates the dispense interface 200 after it has been mounted onto the distal end 42 of the cartridge holder 40 of the drug delivery device 10 illustrated in FIG. 1. A double ended needle 400 is also mounted to the distal end of this interface. The cartridge holder 40 is illustrated as having a first cartridge containing a first medicament and a second cartridge containing a second medicament.

When the interface 200 is first mounted over the distal end of the cartridge holder 40, the proximal piercing end 244 of the first piercing needle 240 pierces the septum of the first cartridge 90 and thereby resides in fluid communication with the primary medicament 92 of the first cartridge 90. A distal end of the first piercing needle 240 will also be in fluid communication with a first fluid path groove 264 defined by the valve seal 260.

Similarly, the proximal piercing end 254 of the second piercing needle 250 pierces the septum of the second cartridge 100 and thereby resides in fluid communication with the secondary medicament 102 of the second cartridge 100. A distal end of this second piercing needle 250 will also be in fluid communication with a second fluid path groove 266 defined by the valve seal 260.

FIG. 11 illustrates a preferred arrangement of such a dispense interface 200 that is coupled to a distal end 15 of the main body 14 of drug delivery device 10. Preferably, such a dispense interface 200 is removably coupled to the cartridge holder 40 of the drug delivery device 10.

As illustrated in FIG. 11, the dispense interface 200 is coupled to the distal end of a cartridge housing 40. This cartridge holder 40 is illustrated as containing the first cartridge 90 containing the primary medicament 92 and the second cartridge 100 containing the secondary medicament 102. Once coupled to the cartridge housing 40, the dispense interface 200 essentially provides a mechanism for providing a fluid communication path from the first and second cartridges 90, 100 to the common holding chamber 280. This holding chamber 280 is illustrated as being in fluid communication with a dose dispenser. Here, as illustrated, this dose dispenser comprises the double ended needle assembly 400. As illustrated, the proximal end of the double ended needle assembly is in fluid communication with the chamber 280.

In one preferred arrangement, the dispense interface is configured so that it attaches to the main body in only one orientation, that is it is fitted only one way round. As such as illustrated in FIG. 11, once the dispense interface 200 is attached to the cartridge holder 40, the primary needle 240 can only be used for fluid communication with the primary medicament 92 of the first cartridge 90 and the interface 200 would be prevented from being reattached to the holder 40 so that the primary needle 240 could now be used for fluid communication with the secondary medicament 102 of the second cartridge 100. Such a one way around connecting mechanism may help to reduce potential cross contamination between the two medicaments 92 and 102.

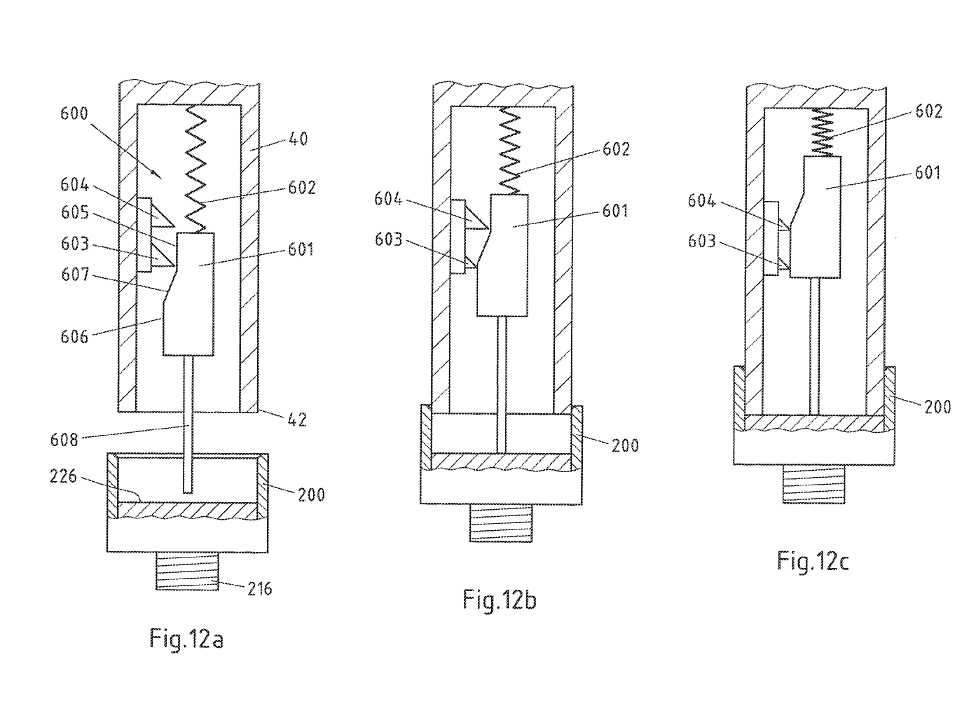

FIG. 12a to c illustrate a cross-sectional view of the attaching of the dispense interface 200 onto the drug delivery device 10. The drug delivery device 10 comprises a detecting arrangement 600 comprising a push rod 601. For instance, the detecting arrangement 600 is at least partially arranged in a cavity formed by the cartridge holder 40 such as cavity 43 in FIG. 11.

At the proximal end of the push rod 601, a spring 602 is arranged which is connected to the cartridge holder 40 such that the push rod 601 is resiliently hold in the drug delivery device 10 and is at least longitudinally movable in the drug delivery device.

The detecting arrangement 600 further comprises a first switch 603 and a second switch 604 which are longitudinally arranged at a side-wall of the cavity 43. Therein, the first switch 603 is arranged closer to the distal end 42 of the cartridge holder 40 than the second switch. In other words, the first switch 603 is distally positioned and the second switch 604 is proximally positioned in the drug delivery device 10. The first switch 603 and the second switch 604 are pressure activated switches forming a first and a second detecting unit. In particular, the first switch 603 and the second switch 604 are only activated, when pressure is applied on the respective switch, and otherwise deactivated. The switches may be connected to a micro-processor control unit of the drug delivery device 10, for instance logically signalling activation and deactivation to the micro-processor control unit.

A lateral surface of the push rod 601 oriented towards the first switch 603 and the second switch 604 is formed from three portions, two parallel surface portions 605, 606 and an inclined surface portion 607. The inclined surface portion 607 is arranged between the parallel surface portions 605, 606 such that the parallel surface portion 605 at the proximal end of the push rod is set back. A rod 608 is arranged at the distal end of the push rod 601.

In FIG. 12a, the dispense interface 200 is not attached to the drug delivery device 10. In particular, there is no contact between the rod 608 and the surface 226 of the dispense interface 200. Accordingly, the spring 602 is relaxed or held with a low pressure and the push rod 601 is held in a first position in the drug delivery device 10. In this first position of the push rod 601 in the drug delivery device 10, the first switch 603 and the second switch 604 face the set back parallel surface portion 605 and the spring 602, respectively. In particular, there is no contact between the lateral surface of the push rod 601 and the first switch 603 and the second switch 604. Both switches are deactivated.

In FIG. 12b, attaching of the dispense interface 200 to the drug delivery device 10 is initiated such that the dispense interface 200 is aligned to the distal end 42 of the cartridge holder 40 and pushed towards the drug delivery device 10 to axially slide over the distal end 42 of the cartridge housing 40 of the drug delivery device 10. Thereby, the distal end of the rod 608 resides on the surface 226 of the dispense interface 200 and is also pushed towards the drug delivery device 10 such that, during attaching the dispense interface 200 to the drug delivery device 10, the movement of the dispense interface 200 towards the drug delivery device facilitates a corresponding movement of the push rod 601 and a compression of the spring 602.

When the push rod 601 is correspondingly moved, the first switch 603 and the second switch 604 slide along the inclined surface portion 607 of the lateral surface of the push rod 601 towards the parallel surface portion 606 and, thereby, increasing pressure is applied on the switches. When a pressure threshold is overcome, the first switch 603 and the second switch 604 are activated, for instance, the switches are activated, when residing on the parallel surface portion 606 (i.e. an activating portion of the push rod). Due to its distal position, the first switch 603 resides on the parallel surface portion 606 before the second switch 604 resides thereon and is, thus, earlier activated. When the attaching is initiated as illustrated in FIG. 12b, the first switch 603 resides on the parallel surface portion 606 and is activated.

In FIG. 12c, attaching of the dispense interface 200 to the drug delivery device 10 is completed such that the septa of the first cartridge 90 and the second cartridge 100 are pierced and the dispense interface resides in fluid communication with the primary medicament 92 of the first cartridge 90 and the secondary medicament 102 of the second cartridge 100 as described above. Furthermore, the protruding members of the cartridge housing (e.g. protruding member 48) may cooperate with the first and second recess 217, 219 of the dispense interface 200 to form a secured mechanical connection such as a snap lock.

When the attaching of the dispense interface 200 to the drug delivery device 10 is completed as illustrated in FIG. 12c, the second switch 604 also resides on the parallel surface portion 606 and is activated. The spring 602 is compressed and the push rod is in a second position.

When the dispense interface 200 is released from the drug delivery device 10, the compressed spring 602 relaxes and moves the push rod 601 back to the first position and optionally the dispense interface 200 to a detent position (i.e. the position illustrated in FIG. 12b). Thereby, firstly the second switch 604 and then the first switch 603 slide along the inclined surface portion 607 towards the set back parallel surface 605 and are subsequently deactivated.

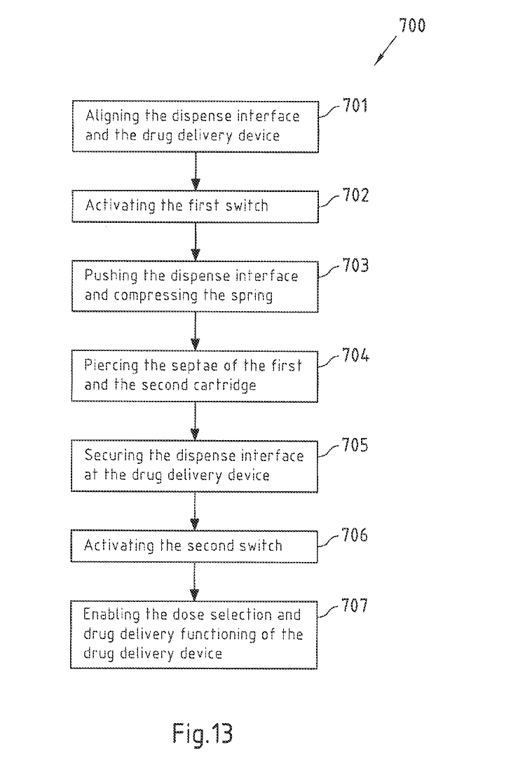

FIG. 13 illustrates a method 700 for attaching the dispense interface 200 to the drug delivery device 10. The steps of the method 700 may at least partially be performed by a micro-processor control unit of the drug delivery device 10.

In a step 701, the dispense interface 200 and the drug delivery device 10 are aligned as illustrated in FIG. 12b. The push rod 601 is already pushed back against the force of the spring. Thereby, in a step 702 the first switch 603 of the detecting arrangement 600 is activated signalling to the micro-processor control unit that attaching of the dispense interface 200 to the drug delivery device 10 is initiated.

In a step 703, the dispense interface 200 is pushed towards the drug delivery device 10 and slid onto the distal end 42 of the cartridge holder 40. Thereby, the movement of the dispense interface 200 facilitates a corresponding movement of the push rod 601 compressing the spring 602.

In a step 704, the piercing needles 240, 250 of the dispense interface 200 pierce the septa of the first and second cartridge 90, 100 of the drug delivery device 10 and reside in fluid communication with the primary medicament 92 and the secondary medicament 102, respectively. The piercing needles and the pierced septa form a fluid connection between the dispense interface 200 and the drug delivery device 10.

In a step 705, the dispense interface 200 is secured at the drug delivery device 10, for instance by a cooperation of the protruding members of the cartridge housing (e.g. protruding member 48) with the first and second recess 217, 219 of the dispense interface 200 forming a secured mechanical connection and, thereby, in a step 706 the second switch 604 of the detecting arrangement 600 is activated signalling to the micro-processor control unit of the drug delivery device that attachment is completed.

In a step 707, the micro-processor control unit checks whether the first switch and the second switch have been subsequently activated and enables the dose selection and drug delivery function of the drug delivery device 10.

For instance, only when both switches are activated the micro-processor control unit will accept this as a complete attaching of the dispense interface 200 to the drug delivery device 10. For instance, when the dispense interface 200 is only partially attached to the drug delivery device 10, the micro-processor control unit will not accept this and not allow the user to activate the dose selection and drug delivery function, because a partial attachment of the hub carries the risk of underdosing the drugs.

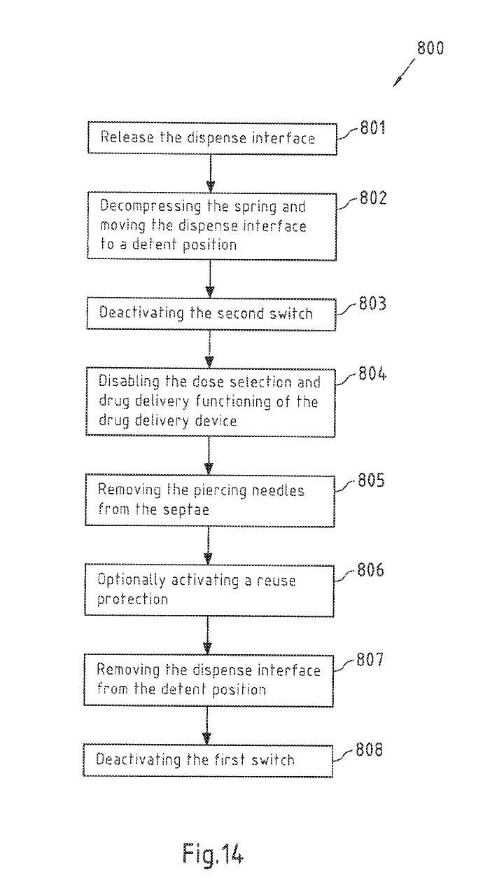

FIG. 14 illustrates a method 800 for removing the dispense interface 200 from the drug delivery device 10. The steps of the method 800 may at least partially be performed by a micro-processor control unit of the drug delivery device 10.

In a first step 801, the secured mechanical connection between the dispense interface 200 and the drug delivery device 10 is released, for instance by pressing a release button or the like.

In a step 802, the spring 602 starts to relax and moves the push rod 601 as the dispense interface 200 is further removed. In an example embodiment, the spring 602 may have sufficient force to move the dispense interface 200 by use of the push rod 601 after the connection to the drug delivery device 10 is released. As the dispense interface 200 is moved to a detent position, the second switch 604 is deactivated in a step 803 signalling to the micro-processor control unit that removing of the dispense interface 200 from the drug delivery device is initiated.

In a step 804, the micro-processor control unit checks whether the first switch 603 and/or the second switch 604 is deactivated and disables the dose selection and drug delivery function of the drug delivery device.

In a step 805, the piercing needles 240, 250 are removed from the septa of the first and second cartridge 90, 100 due to the movement of the dispense interface 200 towards the detent position.

In a step 806, the dispense interface arrives at the detent position and, optionally, a reuse protection of the dispense interface 200 is activated, for instance a lock-out spring arranged at the dispense interface 200 is activated.

In a step 807, the dispense interface is removed by the user from the detent position and, thereby in a step 808, the first switch 603 is deactivated signalling to the micro-processor control unit that removing of the dispense interface 200 from the drug delivery device is completed.

For instance, only by removing the dispense interface 200 past the detent position, the first switch is deactivated and only when both switches are no longer active, the micro-processor control unit will accept this as a complete removing of the dispense interface. Accordingly, it is ensured that the reuse protection is activated before the dose selection and drug delivery function of the drug delivery device may be enabled again. For instance, only after a complete removing and a subsequent complete attaching of a (new) dispense interface, the dose selection and drug delivery function of the drug delivery device 10 may be enabled again.

One or more steps of the methods described in relation to FIG. 13 and FIG. 14 may occur in parallel or in reverse order. For example, in the method of FIG. 13 the step 706 activating the second switch may happen at the same time or (slightly) before the dispense interface 200 is secured at the drug delivery device 10. Similarly, in FIG. 14 the step 805 removing the piercing needles 240,250 from the septa may already take place before deactivating the second switch in step 803.

As described above, there are several risks associated with the partial attaching or removing and reattachment of the dispense interface 200. To effectively mitigate these risks the drug delivery device 10 comprises the detecting arrangement 600. This is inter-alia advantageous to allow to detect whether an attaching and/or removing of the dispense interface 200 is initiated but not completed and to only enable the dose selection and drug delivery function of the drug delivery device, when a complete attaching (and for instance a prior complete removing) is detected.

The term “drug” or “medicament”, as used herein, means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, an enzyme, an antibody or a fragment thereof, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exedin-3 or exedin-4 or an analogue or derivative of exedin-3 or exedin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyhepta-decanoyl) human insulin.

Exendin-4 for example means Exendin-4(1-39), a peptide of the sequence H His- Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:
H-(Lys)4-des Pro36, des Pro37 Exendin-4(1-39)-NH2,
H-(Lys)5-des Pro36, des Pro37 Exendin-4(1-39)-NH2,
des Pro36 [Asp28] Exendin-4(1-39),
des Pro36 [IsoAsp28] Exendin-4(1-39),
des Pro36 [Met(O)14, Asp28] Exendin-4(1-39),
des Pro36 [Met(O)14, IsoAsp28] Exendin-4(1-39),
des Pro36 [Trp(O2)25, Asp28] Exendin-4(1-39),
des Pro36 [Trp(O2)25, IsoAsp28] Exendin-4(1-39),
des Pro36 [Met(O)14 Trp(O2)25, Asp28] Exendin-4(1-39),
des Pro36 [Met(O)14 Trp(O2)25, IsoAsp28] Exendin-4(1-39); or
des Pro36 [Asp28] Exendin-4(1-39),
des Pro36 [IsoAsp28] Exendin-4(1-39),
des Pro36 [Met(O)14, Asp28] Exendin-4(1-39),
des Pro36 [Met(O)14, IsoAsp28] Exendin-4(1-39),
des Pro36 [Trp(O2)25, Asp28] Exendin-4(1-39),
des Pro36 [Trp(O2)25, IsoAsp28] Exendin-4(1-39),
des Pro36 [Met(O)14 Trp(O2)25, Asp28] Exendin-4(1-39),
des Pro36 [Met(O)14 Trp(O2)25, IsoAsp28] Exendin-4(1-39),
   wherein the group -Lys6-NH2 may be bound to the C-terminus of the Exendin-4 derivative;
   or an Exendin-4 derivative of the sequence
H-(Lys)6-des Pro36 [Asp28] Exendin-4(1-39)-Lys6-NH2,
des Asp28 Pro36, Pro37, Pro38Exendin-4(1-39)-NH2,
H-(Lys)6-des Pro36, Pro38 [Asp28] Exendin-4(1-39)-NH2,
H-Asn-(Glu)5des Pro36, Pro37, Pro38 [Asp28] Exendin-4(1-39)-NH2,
des Pro36, Pro37, Pro38 [Asp28] Exendin-4(1-39)-(Lys)6-NH2,
H-(Lys)6-des Pro36, Pro37, Pro38 [Asp28] Exendin-4(1-39)-(Lys)6-NH2,
H-Asn-(Glu)5-des Pro36, Pro37, Pro38 [Asp28] Exendin-4(1-39)-(Lys)6-NH2,
H-(Lys)6-des Pro36 [Trp(O2)25, Asp28] Exendin-4(1-39)-Lys6-NH2,
H-des Asp28 Pro36, Pro37, Pro38 [Trp(O2)25] Exendin-4(1-39)-NH2,
H-(Lys)6-des Pro36, Pro37, Pro38 [Trp(O2)25, Asp28] Exendin-4(1-39)-NH2,
H-Asn-(Glu)5-des Pro36, Pro37, Pro38 [Trp(O2)25, Asp28] Exendin-4(1-39)-NH2,
des Pro36, Pro37, Pro38 [Trp(O2)25, Asp28] Exendin-4(1-39)-(Lys)6-NH2,
H-(Lys)6-des Pro36, Pro37, Pro38 [Trp(O2)25, Asp28] Exendin-4(1-39)-(Lys)6-NH2,
H-Asn-(Glu)5-des Pro36, Pro37, Pro38 [Trp(O2)25, Asp28] Exendin-4(1-39)-(Lys)6-NH2,
H-(Lys)6-des Pro36 [Met(O)14, Asp28] Exendin-4(1-39)-Lys6-NH2,
des Met(O)14 Asp28 Pro36, Pro37, Pro38 Exendin-4(1-39)-NH2,
H-(Lys)6-desPro36, Pro37, Pro38 [Met(O)14, Asp28] Exendin-4(1-39)-NH2,
H-Asn-(Glu)5-des Pro36, Pro37, Pro38 [Met(O)14, Asp28] Exendin-4(1-39)-NH2,
des Pro36, Pro37, Pro38 [Met(O)14, Asp28] Exendin-4(1-39)-(Lys)6-NH2,
H-(Lys)6-des Pro36, Pro37, Pro38 [Met(O)14, Asp28] Exendin-4(1-39)-(Lys)6-NH2,
H-Asn-(Glu)5 des Pro36, Pro37, Pro38 [Met(O)14, Asp28] Exendin-4(1-39)-(Lys)6-NH2,
H-Lys6-des Pro36 [Met(O)14, Trp(O2)25, Asp28] Exendin-4(1-39)-Lys6-NH2,
H-des Asp28 Pro36, Pro37, Pro38 [Met(O)14, Trp(O2)25] Exendin-4(1-39)-NH2,
H-(Lys)6-des Pro36, Pro37, Pro38 [Met(O)14, Asp28] Exendin-4(1-39)-NH2,
H-Asn-(Glu)5-des Pro36, Pro37, Pro38 [Met(O)14, Trp(O2)25, Asp28] Exendin-4(1-39)-NH2,
des Pro36, Pro37, Pro38 [Met(O)14, Trp(O2)25, Asp28] Exendin-4(1-39)-(Lys)6-NH2,
H-(Lys)6-des Pro36, Pro37, Pro38 [Met(O)14, Trp(O2)25, Asp28] Exendin-4(S1-39)-(Lys)6-NH2,
H-Asn-(Glu)5-des Pro36, Pro37, Pro38 [Met(O)14, Trp(O2)25, Asp28] Exendin-4(1-39)-(Lys)6-NH2;
   or a pharmaceutically acceptable salt or solvate of any one of the afore-mentioned Exedin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (˜150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; α and γ contain approximately 450 amino acids and 6 approximately 500 amino acids, while μ and ε have approximately 550 amino acids. Each heavy chain has two regions, the constant region (CH) and the variable region (VH). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H-H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1 C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

1-14. (canceled)

15. An apparatus, comprising:
a detecting arrangement comprising a first and a second detecting unit,
wherein said detecting arrangement is configured to at least detect a partial attaching of an attachable unit to said apparatus and a complete attaching of said attachable unit to said apparatus,
wherein said attachable unit is configured to be removably attached to said apparatus, and
wherein said detecting arrangement is configured to only enable at least one function of said apparatus, when a complete attaching of said attachable unit to said apparatus is detected,
wherein said detecting arrangement is configured to only enable said at least one function of said apparatus after said second detecting unit is deactivated, when subsequently said first detecting unit is deactivated, and when subsequently said first and said second detecting unit are activated.

16. The apparatus according to claim 15,
wherein said first detecting unit is activated, when said attachable unit is partially attached to said apparatus, and
wherein said second detecting unit is activated, when said attachable unit is completely attached to said apparatus.

17. The apparatus according to claim 16, wherein said detecting arrangement is configured to only enable said at least one function of said apparatus, when said first detecting unit and said second detecting unit are activated.

18. The apparatus according to claim 15,
wherein said detecting arrangement is configured to detect a partial removing of said attachable unit from said apparatus and a complete removing of said attachable unit from said apparatus, and
wherein said detecting arrangement is configured to disable said at least one function of said apparatus, when at least said partial removing of said attachable unit from said apparatus is detected.

19. The apparatus according to claim 18,
wherein said second detecting unit is deactivated, when said attachable unit is partially premoved from said apparatus, and
wherein said first detecting unit is deactivated, when said attachable unit is completely removed from said apparatus.

20. The apparatus according to claim 19, wherein said detecting arrangement is configured to disable said at least one function of said apparatus, when said second detecting unit is deactivated.

21. The apparatus according to claim 15, wherein said first and/or said second detecting unit is at least one of an optical sensor, a contact sensor and a proximity sensor.

22. The apparatus according to claim 15, wherein said detecting arrangement is configured to activate a warning, when a partial attaching and/or a partial removing of said attachable unit is detected.

23. The apparatus according claim 15, said detecting arrangement comprising:
a movable element configured to be moved from a first position at said apparatus to a second position at said apparatus during attaching of said attachable unit to said apparatus and to be moved from said second position to said first position during removing of said attachable unit from said apparatus.

24. The apparatus according to claim 23, wherein said movable element (601) is spring loaded at least in said second position.

25. The apparatus according to claim 23,
wherein said first and said second detecting unit are deactivated, when said movable element is in said first position,
wherein said first detecting unit is activated and said second detecting unit is deactivated, when said movable element is positioned between said first and said second position, and
wherein said first and second detecting unit are activated, when said movable element is in said second position.

26. The apparatus according to claim 15, wherein said apparatus is a medical device configured to eject a medicament and/or said attachable unit is a dispense interface or a needle assembly attachable to said medical device.

27. The apparatus according to claim 26, wherein said at least one function of said apparatus is selecting a dose of said medicament and/or ejecting said medicament.

28. A method, comprising:
detecting, by a detecting arrangement comprising a first and a second detecting unit, a partial attaching of an attachable unit to an apparatus and a complete attaching of said attachable unit to said apparatus,
wherein said attachable unit is configured to be removably attached to said apparatus, and
only enabling at least one function of said apparatus, when a complete attaching of said attachable unit to said apparatus is detected.
